(19) United States
(12) Patent Application Publication
(10) Pub. No.: US 2024/0345024 A1
(43) Pub. Date: Oct. 17, 2024

(54) MICROCHIP ELECTROPHORESIS METHOD

(71) Applicant: SHIMADZU CORPORATION, Kyoto-shi (JP)

(72) Inventor: Akihiro ARAI, Kyoto-shi (JP)

(73) Assignee: SHIMADZU CORPORATION, Kyoto-shi (JP)

(21) Appl. No.: 18/614,001

(22) Filed: Mar. 22, 2024

(30) Foreign Application Priority Data
Mar. 23, 2023 (JP) 2023-046973

(51) Int. Cl. G01N 27/447; C12Q 1/6806

(52) U.S. Cl. CPC G01N 27/44791; C12Q 1/6806

(57) ABSTRACT

The present method includes preparation step of preparing an electrophoresis chip, filling step of filling channels with a medium, introduction step of introducing a sample into an introduction channel and applying voltage to the introduction channel, and separation step of applying voltage to a separation channel. The introduction channel includes an upstream side channel and a downstream side channel extending in first direction, and a cross channel extending in second direction. The separation channel shares the cross channel in the middle of the separation channel. In the introduction step, voltage is applied to the separation channel so that potential in the cross channel is higher than potential on the second direction upstream side and the second direction downstream side of the cross channel, and potential gradient on the second direction upstream side of the cross channel is larger than potential gradient on the second direction downstream side thereof.

### CROSS REFERENCE TO RELATED APPLICATIONS

This application claims priority to Japanese Patent Application No. 2023-046973 filed on Mar. 23, 2023, the entire disclosure of which is incorporated by reference herein.

### BACKGROUND OF THE INVENTION

### Field of the Invention

The present invention relates to a microchip electrophoresis method.

### Description of the Related Art

Short-chain RNAs such as sgRNA and miRNA are known to enable genome editing and to serve as a modulator in biochemical processes such as cell proliferation, differentiation and cell cycle. As a measurement method for separating and analyzing DNA containing short-chain RNA, protein, and the like, a capillary electrophoresis method has been conventionally used, but in particular, in recent years, a microchip electrophoresis method is becoming widely used from the viewpoint of being able to perform measurement with a smaller amount of a measurement sample in a shorter period of time.

In the microchip electrophoresis method, for example, a target sample is separated by an electrophoresis method in a channel (groove) having a width and a depth of several ten μm or less formed in a chip having size of several centimeters. As a device used in such a microchip electrophoresis method, for example, JP-A-2017-053726 and the like are provided.

In a conventional microchip electrophoresis method, a channel formed in a chip is short, so that analysis can be performed at high speed. In addition, since only a target sample put in an intersection 23 (see FIG. 10) where a sample introduction channel and a sample separation channel intersect is moved to the downstream side of the sample separation channel (that is, the detector side) and separated, a length of a target sample actually subjected to separation (sample plug) can be shortened, and excellent separability is obtained. On the other hand, since an amount of a target sample to be subjected to separation is small, a problem that the target sample cannot be detected by an analyzer and a problem that detection sensitivity is poor occur. On the other hand, it is considered to improve detection sensitivity by increasing a distance of the intersection and increasing an amount of a target sample to be subjected to separation, but in this case, a length of the target sample is increased and separability is lowered.

### SUMMARY OF THE INVENTION

An object of the present invention is to provide a microchip electrophoresis method that achieves both excellent detection sensitivity and separability.

A microchip electrophoresis method according to a first aspect of the present invention includes, in order, a preparation step of preparing a chip for electrophoresis including a sample introduction channel extending in a first direction and a sample separation channel extending in a second direction intersecting the first direction, a filling step of filling the sample introduction channel and the sample separation channel with a separation medium for electrophoresis, an introduction step of filling the entire sample introduction channel with a sample by introducing the sample into the sample introduction channel and applying voltage to the sample introduction channel so as to generate potential gradient in the first direction in the sample introduction channel, and a separation step of separating a target substance in the sample by applying voltage into the sample separation channel so that potential gradient is generated in the second direction in the sample separation channel. The sample introduction channel includes an upstream side channel extending in the first direction, a cross channel extending in the second direction from a first direction downstream side end portion of the upstream side channel, a downstream side channel extending in the first direction from a second direction downstream side end portion of the cross channel. The sample separation channel shares the cross channel in the middle of the sample separation channel. In the introduction step, voltage is applied to the sample separation channel such that potential in the cross channel is higher than potential on the second direction upstream side and the second direction downstream side of the cross channel, and such that potential gradient on the second direction upstream side of the cross channel is larger than potential gradient on the second direction downstream side of the cross channel.

According to the microchip electrophoresis method of the first aspect, an analysis target can be analyzed with excellent sensitivity and separability.

### BRIEF DESCRIPTION OF THE DRAWINGS

FIG. 1 is a schematic diagram of an electrophoresis device used in a microchip electrophoresis method of a first embodiment;

FIG. 2 is a cross-sectional view of a microchip of the electrophoresis device of FIG. 1;

FIG. 3 is a plan view of a lower transparent substrate of the microchip of FIG. 2 and a partially enlarged view of the lower transparent substrate;

FIG. 4 is a diagram (left diagram) schematically illustrating movement of a sample during an introduction step, and a diagram (right diagram) expressing current flowing through each channel by Kirchhoff's laws;

FIG. 5 is a diagram (left diagram) schematically illustrating movement of a sample during a separation step, and a diagram (right diagram) expressing current flowing through each channel by Kirchhoff's laws;

FIG. 6 is a plan view of the lower transparent substrate of the microchip according to another embodiment;

FIG. 7 is an electropherogram obtained by performing the electrophoresis method of the first embodiment;

FIG. 8 illustrates peak heights of electropherograms in an example and a comparative example, where the horizontal axis shows a difference in potential gradient (E₃-E₄), and the vertical axis shows a peak height;

FIG. 9 illustrates the number of theoretical plates when potential gradient E′ on the upstream side in a first direction and potential gradient E′2 on the downstream side in the first direction during the separation step are changed, where the horizontal axis represents the potential gradients E′1 and E′2, and the vertical axis represents the number of theoretical plates; and

FIG. 10 is a plan view of a lower transparent substrate of a microchip of a conventional electrophoresis device.

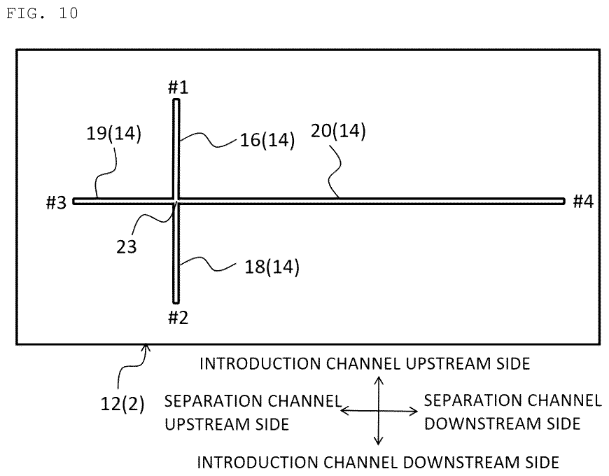

### DETAILED DESCRIPTION OF THE PREFERRED EMBODIMENTS

### 1. First Embodiment

A microchip electrophoresis method according to a first embodiment of the present invention is a method for analyzing short-chain RNA as a target substance. This method includes a preparation step, a filling step, a thermal denaturation step, an introduction step, and a separation step in order.

### (Preparation Step)

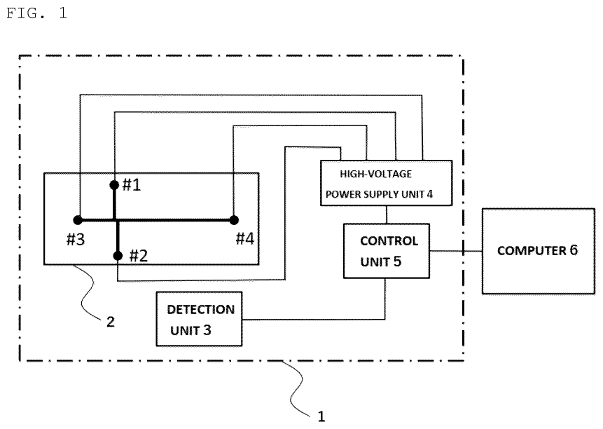

In the preparation step, a microchip electrophoresis device 1 is prepared. As illustrated in FIG. 1, the microchip electrophoresis device 1 includes a microchip 2, a detection unit 3, a high-voltage power supply unit 4, and a control unit 5.

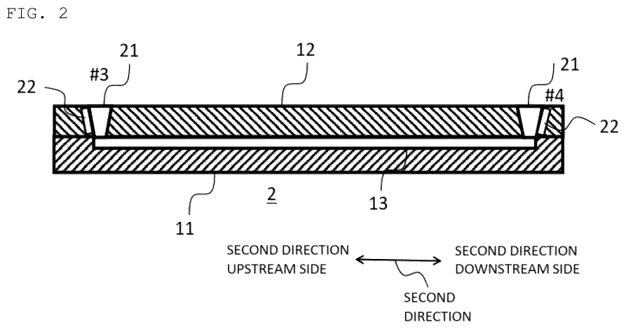

As illustrated in FIG. 2, the microchip 2 includes a pair of upper and lower transparent substrates, that is, a lower transparent substrate 11 and an upper transparent substrate 12. A channel (groove) 13 is formed on an upper surface of the lower transparent substrate 11. The channel 13 includes a sample introduction channel 14 extending in a first direction and a sample separation channel 15 extending in a second direction orthogonal to the first direction.

The sample introduction channel 14 includes a first upstream side channel 16, a cross channel 17, and a first downstream side channel 18 in this order. The first upstream side channel 16 is located on the first direction upstream side of the cross channel 17, and extends from a first direction upstream side end portion (end portion on the upstream side in the first direction) of the sample introduction channel 14 toward the first direction downstream side to a second direction upstream side end portion of the cross channel 17. The cross channel 17 is a shared portion between the sample introduction channel 14 and the sample separation channel 15, and extends from a first direction downstream side end portion of the first upstream side channel 16 toward the second direction downstream side. The first downstream side channel 18 is located on the first direction downstream side of the cross channel 17, and extends from a second direction downstream side end portion of the cross channel 17 toward the first direction downstream side to the first direction downstream side end portion of the sample introduction channel 14. That is, the cross channel 17 is connected to the first upstream side channel 16 at a second direction upstream side end portion of the cross channel 17, and is connected to the first downstream side channel 18 at a second direction downstream side end portion of the cross channel 17. The sample introduction channel 14 has a Z shape in which two angles are perpendicular to each other. A first direction upstream side end portion of the sample introduction channel 14, that is, a first direction upstream side end portion of the first upstream side channel 16 constitutes a port #1. In the port #1, a sample and a separation medium are introduced, and after analysis, the sample and the separation medium are discharged. A first direction downstream side end portion of the sample introduction channel 14, that is, a first direction downstream side end portion of the first downstream side channel 18 constitutes a port #2. In the port #2, a separation medium is introduced as necessary, and after analysis, a sample and the separation medium are discharged.

The sample separation channel 15 extends linearly in the second direction from its second direction upstream side end portion to its second direction downstream side end portion, and shares the cross channel 17 in the middle of the sample separation channel 15. Specifically, the sample separation channel 15 includes a second upstream side channel 19, the cross channel 17, and a second downstream side channel 20 in this order. The second upstream side channel 19 is located on the second direction upstream side of the cross channel 17, and extends from a second direction upstream side end portion of the sample separation channel 15 toward the second direction downstream side to a second direction upstream side end portion of the cross channel 17. The second downstream side channel 20 is located on the second direction downstream side of the cross channel 17, and extends from a second direction downstream side end portion of the cross channel 17 toward the second direction downstream side to a second direction downstream side end portion. In the second downstream side channel 20, separation of a sample occurs and is detected by a detection unit to be described later. A second direction upstream side end portion of the second upstream side channel 19 constitutes a port #3. In the port #3, a separation medium is introduced, and after analysis, a sample and the separation medium are discharged. A second direction downstream side end portion of the second downstream side channel 20 constitutes a port #4. In the port #4, a separation medium is introduced as necessary, and after analysis, a sample and the separation medium are discharged.

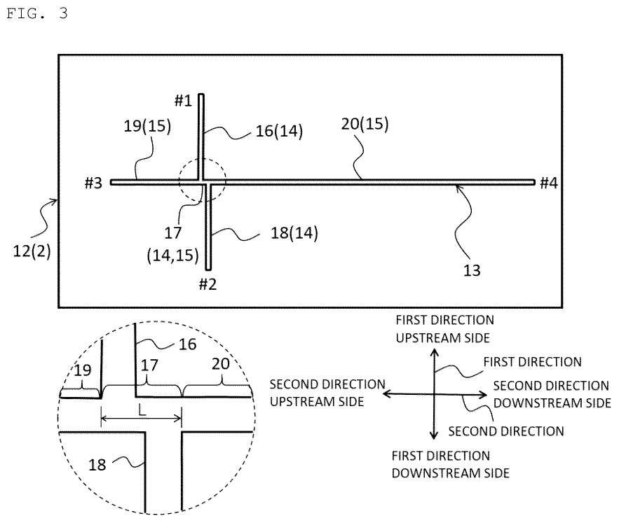

A width (a second direction distance between the first upstream side channel 16 and the first downstream side channel 18, a first direction distance between the second upstream side channel 19, the cross channel 17 and the second downstream side channel 20) and a depth of each of the channels 13 are, for example, 10 μm or more, preferably 30 μm or more, and are, for example, 200 μm or less, preferably 150 μm or less. A length L (a second direction distance from a second direction upstream end edge to a second direction downstream end edge in a shared portion between the sample introduction channel 14 and the sample separation channel 15, see FIG. 3) of the cross channel 17 is longer than a width of the channel, for example, 100 μm or more, preferably 150 μm or more, and for example, 500 μm or less, preferably 300 μm or less. A length of each of the first upstream side channel 16, the first downstream side channel 18, and the second upstream side channel 19 is, for example, 1 mm or more and 10 mm or less. A length of the second downstream side channel is 10 mm or more and 100 mm or less, for example. Lengths of the first upstream side channel 16, the first downstream side channel 18 and the second upstream side channel 19 are preferably substantially the same. The term “substantially the same” means that, for example, a difference between them is 10% or less.

In the upper transparent substrate 12, a through hole 21 penetrating in a vertical direction is formed at a position corresponding to each of the ports #1 to #4 (see FIG. 2). The through hole 21 is a reservoir for introducing into a channel and storing a sample and/or a separation medium.

In the microchip 2, an electrode element 22 is arranged around each of the ports #1 to #4 so that desired voltage can be applied to each of the ports.

A pair of transparent substrates are made from glass such as quartz glass or borosilicate glass, for example, and, in addition, transparent resin or the like.

The detection unit 3 is an element that detects a sample separated by the sample separation channel 15 in the chip 2. Specific examples include a fluorescence measurement device that measures fluorescence generated by irradiation of a sample component with excitation light, an absorption measurement device that measures absorbance by irradiation with light, and a device that measures chemiluminescence or bioluminescence of a sample component that is electrically isolated. A fluorescence measurement device is preferable. In a case where the detection unit 3 is a fluorescence measurement device, the detection unit includes a light source (for example, a light emitting diode), a filter that transmits desired fluorescence, a photomultiplier tube that receives fluorescence, and the like.

As illustrated in FIG. 1, the high-voltage power supply unit 4 is electrically connected to each of the electrode elements 22 provided in the ports #1 to #4 through each wiring. Desired voltage can be independently applied to each of the electrode elements 22.

The control unit 5 is an element that controls operation of an electrophoresis method and the high-voltage power supply unit 4. For example, an amount of a sample introduced into a channel and an ionization medium is adjusted, or an amount of voltage applied to a port is adjusted in a high-voltage power supply. The control unit 5 is realized by, for example, a microcomputer equipped with a central processing unit, a storage device, and the like, and is connected to a personal computer 1 equipped with predetermined software.

In addition to the above component, the microchip electrophoresis device 1 may include other elements for performing or assisting electrophoresis, for example, a dispensing probe element for injecting a sample and an ionization medium into a port, a suction nozzle for sucking a sample and an ionization medium injected into a port, and the like. For these, reference can be made to, for example, JP-A-2020-128904.

### (Filling Step)

In the filling step, the sample introduction channel and the sample separation channel are filled with a separation medium. Specifically, a separation medium is introduced into a through hole corresponding to the ports #1 to #4, and channels are filled with the separation medium.

The separation medium is a separation medium that can be used for electrophoresis, and examples of the separation medium include a buffer solution containing water-soluble cellulose. Preferably, a buffer solution containing water-soluble cellulose and sugar alcohol is used. This makes it possible to improve separability of short-chain RNA without increasing viscosity of a separation medium. Note that, other than the above, a polyacrylamide solution or the like can also be used as a separation medium.

Examples of the buffer solution include a TBE buffer solution, a TAE buffer solution, a TE buffer solution, a phosphate buffer solution, and acetic acid-sodium acetate buffer solution.

Examples of the water-soluble cellulose include alkyl cellulose such as methyl cellulose, ethyl cellulose, and propyl cellulose; hydroxyalkyl cellulose such as hydroxymethyl cellulose and hydroxyethyl cellulose; and carboxyalkyl cellulose such as carboxymethyl cellulose and carboxyethyl cellulose. Preferably, hydroxyalkyl cellulose is used. Concentration of the water-soluble cellulose in a separation medium is, for example, 0.1 w/v % or more, preferably 0.5 w/v % or more, and is, for example, 5 w/v % or less, preferably 3 w/v % or less.

Examples of the sugar alcohol include sorbitol, mannitol, maltitol, erythritol, xylitol, lactitol, and the like. Preferably, sorbitol is used. Concentration of the sugar alcohol in a separation medium is, for example, 0.5 w/v % or more, preferably 1 w/v % or more, and is, for example, 5 w/v % or less, preferably 3 w/v % or less. In addition to or in place of the sugar alcohol, polysaccharide such as pullulan, agarose, and dextrin can also be contained.

When fluorescence analysis is performed using a fluorescence measurement device as a detection unit, for example, fluorescent dye that is combined with short-chain RNA is added to a separation medium. Examples of such dye include GelStar (registered trademark, manufactured by Lonza K.K.), SYBR Gold (registered trademark, manufactured by Thermo Fisher Scientific Inc.), SYBR Green II (registered trademark, manufactured by Thermo Fisher Scientific Inc.), and the like.

### (Thermal Denaturation Step)

In the thermal denaturation step, a sample is heated and a target substance in the sample is thermally denatured. Specifically, a sample solution is prepared, and the sample solution is heated for a predetermined period of time and then slowly cooled. By the above, a structure of the target substance in the sample can be denatured.

The sample includes a biologically derived sample containing short-chain RNA which is a substance to be measured. Specifically, examples of the sample include a body fluid such as blood, cerebrospinal fluid, urine, body secretion, saliva, sputum, or feces; a biological tissue, a cell, and the like. The sample may be one in a state of containing an impurity collected from a living body, or may be one in a state where an impurity is removed by pretreatment such as purification. A length of short-chain RNA as a target substance of measurement is, for example, 200 nt or less, and is, for example, 10 nt or more. That is, in order to separate and analyze RNA having 200 nt or less, it is preferable to perform the electrophoresis method of the first embodiment. The short-chain RNA may be either single-stranded RNA or double-stranded RNA, but from the viewpoint of expansion of an RNA structure by heating, it is preferable to use single-stranded RNA as a target substance of measurement.

A sample solution is obtained by mixing a sample with a buffer solution. Examples of the buffer solution include one similar to the buffer solution exemplified above. Further, an internal standard marker may be added to a sample together with the buffer solution. Examples of an internal standard marker-containing buffer include a marker solution contained in a reagent kit (DNA-50 kit, DNA-500 kit, DNA-12000 kit; manufactured by Shimadzu Corporation) for MCE-202 MultiNA (registered trademark), which is a microchip electrophoresis device for DNA/RNA analysis.

A heating temperature of a sample solution is, for example, 50° C. or more, preferably 60° C. or more, and is, for example, 95° C. or less, preferably 80° C. or less. Heating time is, for example, 1 minute or more, preferably 3 minutes or more, and is, for example, 30 minutes or less, preferably 10 minutes or less. Since a structure of folded short-chain RNA is linearly expanded by heat treatment, a band derived from a secondary structure is eliminated, and mobility can be separated according to a base length.

Slow cooling is performed after heating, for example, by using a temperature adjustment device such as a thermostatic bath. Temperature after slow cooling is, for example, 10° C. or less, and is, for example, 0° C. or more. After slow cooling, it is preferable to introduce a sample solution into a channel immediately and perform electrophoresis. Specifically, it is preferable to perform electrophoresis within five minutes after a target slow cooling temperature is reached. By the above, returning to a folded structure due to disappearance of an expansion effect caused by heating can be prevented.

Through this step, a structure of a target substance in a sample can be denatured. That is, a secondary structure of short-chain RNA can be made straight-chain.

### (Introduction Step)

In the introduction step, a sample is introduced into the sample introduction channel, and voltage is applied to the sample introduction channel so that potential gradient is generated in the first direction.

Specifically, a sample solution is injected into the port #1 through a through hole, and a voltage V₁ and a voltage V₂ are applied to the port #1 and the port #2, respectively. At this time, the voltages V₁ and V₂ are set such that potential P₂ (that is, the applied voltage V₂) of the port #2 is higher than potential P₁ (that is, the applied voltage V₁) of the port #1. By the above, since a target substance which is a nucleic acid such as RNA is negatively charged, the target substance can move from the port #1 having low potential to the port #2 having high potential.

The voltage V₁ applied to the port #1 is, for example, 100 V or more, preferably 200 V or more, and is, for example, 400 V or less, preferably 300 V or less. Potential gradient (that is, potential gradient in the first upstream side channel including the port #1) E₁ on the first direction upstream side at this time is, for example, 50 V/cm or more, preferably 150 V/cm or more, and is, for example, 400 V/cm or less, preferably 300 V/cm or less. The potential gradient is calculated by dividing a potential difference of a channel by a length of the channel.

The voltage V₂ applied to the port #2 is higher than the voltage V₁, and is, for example, 400 V or more, preferably 500 V or more, and for example, 800 V or less, preferably 600 V or less. A difference between the voltage V₂ and the voltage V₁ is, for example, 100 V or more, preferably 200 V or more, and is, for example, 500 V or less, preferably 400 V or less. Potential gradient (that is, potential gradient in the second downstream side channel including the port #2) E₂ on the first direction downstream side at this time is, for example, 200 V/cm or more, preferably 300 V/cm or more, and is, for example, 800 V/cm or less, preferably 600 V/cm or less.

In addition to voltage setting for the ports #1 and #2, voltage is further applied to the sample separation channel so that potential Px in the cross channel is higher than both potential on the second direction upstream side of the cross channel (in the second upstream side channel) and potential on the second direction downstream side of the cross channel (in the second downstream side channel). That is, voltages V₃ and V₄ are set such that the potential Px of the cross channel is higher than potential P₃ of the port #3 and potential P₄ of the port #4. Specifically, the voltage V₃ is applied to the port #3, and the voltage V₄ is applied to the port #4 or the port #4 is grounded. At this time, the voltages V₃ and V₄ are also set such that the potential Px of the cross channel is higher than the potential P₁ of the port #1 and lower than the potential P₂ of the port #2. By the above, it is possible to prevent a sample put in the cross channel from being dispersed to the outer side in the second direction and to keep an appropriate amount in the cross channel.

Note that the potential Px of the cross channel can be calculated by Kirchhoff's laws. That is, currents flowing through channels (the first upstream side channel, the first downstream side channel, the second upstream side channel, and the second downstream side channel) are denoted by I₁ to I₄, and resistances of the channels are denoted by R₁ to R₄. In each channel, since all channels are filled with a uniform separation medium, a resistance value can be assumed to be proportional to a length of the channel, and when lengths of the channels are L₁ to L₄, the resistance values R₁, R₂, R₃, and R₄ of the channels are L₁×K, L₂×K, L₃×K, and L₄×K, respectively. K is a resistance value per unit length common to all channels.

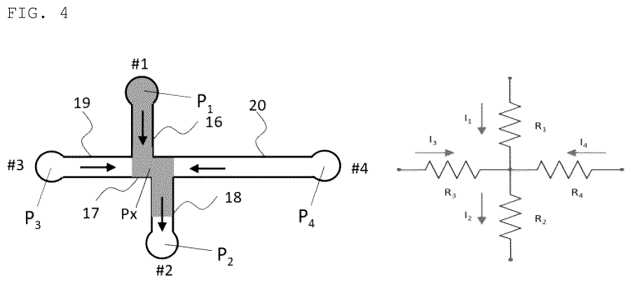

FIG. 4 illustrates a schematic diagram of a direction of current flowing through each channel. From Kirchhoff's laws, Equation (1) below is established, and Equation (2) is derived from Equation (1), and Px can be calculated by calculating Equation (2). With reference to the calculated Px, the potentials P₁ to P₄ of the ports #1 to #4 may be set according to each channel. That is, the voltages V₁ to V₄ corresponding to the potentials P₁ to P₄ may be applied to the ports #1 to #4.

$$[ Equation 1 ]$$

$${I}_{1} + {I}_{3} + {I}_{4} = {I}_{2} \qquad ( 1 )$$

$$\frac{Px - {P}_{1}}{{R}_{1}} + \frac{Px - {P}_{3}}{{R}_{3}} + \frac{Px - {P}_{4}}{{R}_{4}} = \frac{{P}_{2} - Px}{{R}_{2}} \qquad ( 2 )$$

In the first embodiment, from the viewpoint of being able to evenly fill the first upstream side channel, the first downstream side channel and the second upstream side channel by pressurizing and injecting a separation medium from the port #4, lengths of the first upstream side channel, the first downstream side channel and the second upstream side channel are preferably substantially the same, and a length of the second downstream side channel is preferably n times (n is, for example, 5 or more and 15 or less) that of the first upstream side channel. In this case, L₁=L₂=L₃=n×L₄, and Equation (2) is calculated as Equation (3) below.

$$[ Equation 2 ]$$

$$Px = \frac{n}{3 n + 1} ( {P}_{1} + {P}_{2} + {P}_{3} ) + \frac{1}{3 n + 1} {P}_{4} \qquad ( 3 )$$

The applied voltage V₃ is, for example, 150 V or more, preferably 250 V or more, and is, for example, 500 V or less, preferably 400 V or less. Potential gradient (that is, potential gradient in the second upstream side channel including the port #3) E₃ on the second direction upstream side at this time is, for example, 100 V/cm or more, preferably 130 V/cm or more, and is, for example, 500 V/cm or less, preferably 300 V/cm or less.

The applied voltage V₄ is, for example, 100 V or less, preferably 50 V or less, and most preferably 0 V. That is, the port #4 is preferably grounded. Potential gradient (that is, potential gradient in the second downstream side channel including the port #4) E₄ on the second direction downstream side at this time is, for example, 50 V/cm or more, preferably 80 V/cm or more, and is, for example, 200 V/cm or less, preferably 130 V/cm or less.

In the first embodiment, in particular, the potential gradient E₃ on the second direction upstream side is set to be larger than the potential gradient E₄ on the second direction downstream side. A difference in potential gradient between the second direction upstream side and the second direction downstream side in the cross channel at this time is, for example, 10 V/cm or more, preferably 20 V/cm or more, and is, for example, 100 V/cm or less, preferably 70 V/cm or less. By the above, a sample put in the cross channel moves to a second direction downstream side end portion in the cross channel, and can be unevenly distributed as a high-concentration sample aggregate condensed in the second direction. For this reason, separability can be improved while sensitivity is excellent.

Through this step, the entire sample introduction channel, particularly the cross channel, is filled with a sample. Further, in the sample separation channel, only the cross channel is filled with a sample, and diffusion of the sample to the second upstream side channel and the second downstream side channel is prevented.

### (Separation Step)

In the separation step, voltage is applied to the sample separation channel so that potential gradient is generated in the second direction in the sample separation channel.

Specifically, the voltages V₃ and V₄ applied to the ports #3 and #4 are changed, and voltages V′₃ and V′₄ are applied. At this time, each voltage is adjusted so that potential P′₄ of the port #4 (that is, the applied voltage V′₄) is higher than the potential P′₃ of the port #3 (that is, the applied voltage V′₃). By the above, a sample in the cross channel moves toward the port #4 having high potential.

The voltage V's is, for example, 100 V or less, preferably 50 V or less, and most preferably 0 V (ground). Potential gradient E′₃ on the second direction upstream side at this time is, for example, 300 V/cm or more, preferably 400 V/cm or more, and is, for example, 800 V/cm or less, preferably 600 V/cm or less.

The voltage V′₄ is, for example, 500 V or more, preferably 800 V or more, and is, for example, 2000 V or less, preferably 1500 V or less. Potential gradient E′₄ on the second direction downstream side at this time is, for example, 100 V/cm or more, preferably 200 V/cm or more, and is, for example, 500 V/cm or less, preferably 350 V/cm or less.

At this time, in addition to voltage setting of the ports #3 and #4, voltage of the sample introduction channel is further adjusted such that the potentials P₁ and P₂ of the ports #1 and #2 are higher than potential P′x of the cross channel. Specifically, the voltages V₁ and V₂ applied to the ports #1 and #2 are changed to the voltages V′₁ and V′₂, respectively. By the above, a sample put in the upstream side channel and the downstream side channel can be returned to the port #3 or #4, so that the sample can be prevented from continuing to flow to the cross channel or the second downstream side channel even in the separation step, and excellent separability (in particular, number of theoretical plates) can be obtained.

The potential P′x of the cross channel at this time can be calculated by Kirchhoff's laws. That is, currents flowing through channels (the first upstream side channel, the first downstream side channel, the second upstream side channel and the second downstream side channel) are denoted by I′₁ to I′₄, and the other configurations are the same as those in the introduction step.

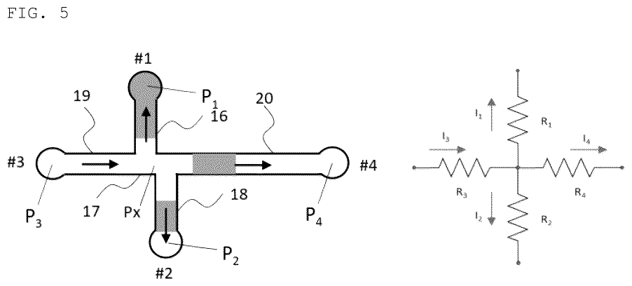

FIG. 5 illustrates a schematic diagram of a direction of current flowing through each channel. From Kirchhoff's laws, Equation (4) below is established, and Equation (5) is derived from Equation (4), and P′x can be calculated by calculating Equation (5). With reference to the calculated P′x, potentials P′₁ to P′₄ of the ports #1 to #4 may be set according to each channel. That is, the voltages V′₁ to V′₄ corresponding to the potentials P′₁ to P′₄ may be applied to the ports #1 to #4.

$$[ Equation 3 ]$$

$${I}_{3}^{′} = {I}_{1}^{′} + {I}_{2}^{′} + {I}_{4}^{′} \qquad ( 4 )$$

$$\frac{{P}^{′} x - {P}_{3}^{′}}{{R}_{3}} = \frac{{P}_{1}^{′} - {P}^{′} x}{{R}_{1}} + \frac{{P}_{2}^{′} - {P}^{′} x}{{R}_{2}} + \frac{{P}_{4}^{′} - {P}^{′} x}{{R}_{4}} \qquad ( 5 )$$

Similarly to Equation (3), when a relationship between lengths of channels is set to a preferable aspect, Equation (5) is calculated as Equation (6) below.

$$[ Equation 4 ]$$

$${P}^{′} x = \frac{n}{3 n + 1} ( {P}_{1}^{′} + {P}_{2}^{′} + {P}_{3}^{′} ) + \frac{1}{3 n + 1} {P}_{4}^{′} \qquad ( 6 )$$

Each of the applied voltage V′₁ and voltage V′₂ is independently, for example, 100 V or more, preferably 200 V or more, and for example, 500 V or less, preferably 350 V or less. Further, the potential gradient E′₁ on the first direction upstream side and the potential gradient E′₂ on the first direction downstream side are lower than the potential gradient E′₄ on the second direction downstream side, and E′₁ or E′₂ is, for example, 100 V/cm or more, preferably 165 V/cm or more, and is, for example, 250 V/cm or less, preferably 185 V/cm or less. A difference between E′₁ (or E′₂) and E′₄ is, for example, 30 V/cm or more, preferably 50 V/cm or more, and is, for example, 150 V/cm or less, preferably 100 V/cm or less. By setting the potential gradients E′₁ and E′₂ within the above range, the number of theoretical plates is improved.

Through this step, a target substance in a sample is separated in the second downstream side channel while moving toward the second downstream side channel of the sample separation channel. Then, the separated target substance is detected by the detection unit and output as an electropherogram. Specifically, since a speed of movement in the sample separation channel varies depending on a type (specifically, a base length) of a target substance, time (migration time) for reaching a detection location detected by the detection unit in the sample separation channel varies. Therefore, for example, in a case of fluorescence analysis, it is possible to analyze a type and an amount of a target substance contained in a sample by measuring reaching time to a detection location, and emission intensity or signal intensity of a fluorescent substance adsorbed to the target substance detected in the reaching time, and analyzing an electropherogram in which they are output.

According to the microchip electrophoresis method of the first embodiment, it is possible to analyze a target sample, particularly short-chain RNA, with excellent sensitivity and excellent separability. In the conventional microchip electrophoresis method without the cross channel, since a sample subjected to separation is only a cross portion (that is, corresponding to a width of an introduction channel), a filling amount of the sample has been small, a sufficient amount for detection has not been secured, and sensitivity has been low. In contrast, in the microchip electrophoresis method including the cross channel in which a length of a cross portion is increased, since a large amount of a target sample can be put in the cross channel and subjected to separation, detection sensitivity of a target sample, and thus sensitivity, is improved. However, since a length of a target sample flowing through the sample introduction channel becomes large, separability (the number of theoretical plates required) becomes low. In order to solve these problem, in the first embodiment, it has been found that the microchip including the cross channel is prepared, and in the introduction step, potential in the cross channel is set to be higher than potential on the second direction upstream side and the second direction downstream side of the cross channel, and then potential gradient on the second direction upstream side of the cross channel is set to be larger than potential gradient on the second direction downstream side of the cross channel. By the above, a large amount of a target sample can be put in the cross channel. In addition, in the introduction step, since a sample in the cross channel moves to the second direction downstream side by the setting of potential gradient as described above, the sample can be unevenly distributed in a minute region at the second direction downstream end portion of the cross channel. That is, before start of separation, aggregate of a sample (sample plug) having high concentration and a short second direction length can be arranged at the second direction downstream side end portion of the cross channel. Therefore, it is possible to achieve both excellent detection sensitivity and separability.

### 2. Other Embodiments

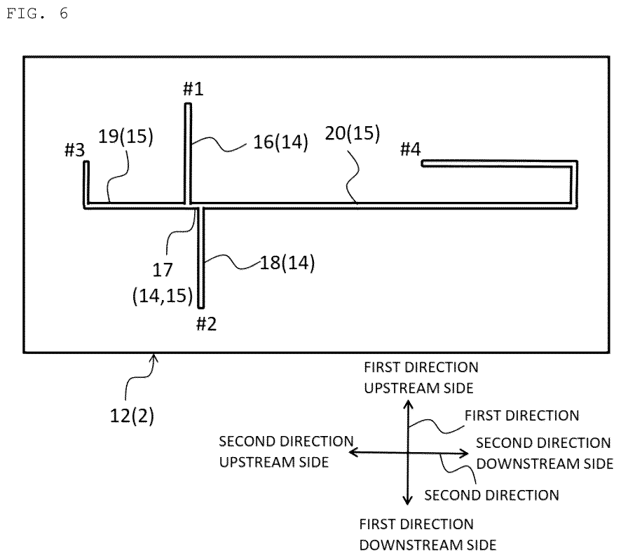

In the first embodiment, the sample separation channel may be folded or bent at its second direction upstream side end portion and second direction downstream side end portion as illustrated in FIG. 6. Further, although not illustrated, the sample introduction channel may also be folded or bent at its first direction upstream side end portion and its first direction downstream side end portion.

In the first embodiment, the preparation step, the filling step, the heating step, the introduction step, and the separation step are performed in order, but for example, the heating step may be performed before the preparation step or the filling step.

In the first embodiment, the first direction and the second direction are orthogonal to each other, but for example, the first direction and the second direction may have, for example, an intersecting direction of 45 degrees or more and less than 90 degrees instead of being orthogonal to each other. In other words, the first upstream side channel and the first downstream side channel may obliquely intersect the second upstream side channel and the second downstream side channel.

### 3. Aspect

It is understood by those skilled in the art that the exemplary embodiment described above is a specific example of an aspect below.

(Clause 1) A microchip electrophoresis method according to one aspect may include:
in order,
a preparation step of preparing a chip for electrophoresis including a sample introduction channel extending in a first direction and a sample separation channel extending in a second direction orthogonal to the first direction;
a filling step of filling the sample introduction channel and the sample separation channel with a separation medium for electrophoresis;
an introduction step of introducing a sample into the sample introduction channel and applying voltage to the sample introduction channel so as to generate potential gradient in the first direction in the sample introduction channel, and thus filling the entire sample introduction channel with the sample; and
a separation step of separating a target substance in the sample by applying voltage into the sample separation channel so that potential gradient is generated in the second direction in the sample separation channel,
in which the sample introduction channel includes:
an upstream side channel extending in the first direction;
a cross channel extending in the second direction from a first direction downstream side end portion of the upstream side channel; and
a downstream side channel extending in the first direction from a second direction downstream side end portion of the cross channel,
the sample separation channel shares the cross channel in the middle of the sample separation channel, and
in the introduction step, voltage may be applied to the sample separation channel such that potential in the cross channel is higher than potential on the second direction upstream side and the second direction downstream side of the cross channel, and such that potential gradient on the second direction upstream side of the cross channel is larger than potential gradient on the second direction downstream side of the cross channel.

(Clause 2) In the microchip electrophoresis method according to Clause 1, in the introduction step, a difference between potential gradient on the second direction upstream side of the cross channel and potential gradient on the second direction downstream side of the cross channel may be set to 20 V/cm or more and 70 V/cm or less.

(Clause 3) In the microchip electrophoresis method according to Clause 1 or 2, the target substance may be short-chain RNA, and the microchip electrophoresis method may further include a thermal denaturation step to perform heating so that the target substance becomes straight-chain before the introduction step.

(Clause 4) In the microchip electrophoresis method according to any one of Clauses 1 to 3, the separation medium for electrophoresis may contain a water-soluble cellulose derivative and sugar alcohol.

### EXAMPLES

Next, the present invention will be described in detail with reference to an example and a comparative example, but the scope of the present invention is not limited to this.

### Example 1

As a sample, 14 ng/μL of an RNA sample (DynaMarker RNA LowII, manufactured by BioDynamics Laboratory Inc.) was used. This sample solution was heat-treated at 65° C. for five minutes immediately before analysis, and then slowly cooled to 4° C. over five minutes. A separation medium was prepared by adding 2% (w/v) of hydroxypropylmethyl cellulose and 1.5% (w/v) of D-sorbitol to 2×TBE buffer. Note that, to this separation medium, fluorescent dye (trade name: GelStar, manufactured by Lonza K.K.) was added so as to have a concentration of 1/20000, and mixed.

As a microchip electrophoresis device, a microchip electrophoresis device for DNA/RNA analysis (trade name: “MultiNA”, manufactured by Shimadzu Corporation) was used. Note that a channel of a microchip had a shape illustrated in FIG. 3. The length L₁ of the first upstream side channel, the length L₂ of the first downstream side channel and the length L₃ of the second upstream side channel were all set to 3.62 mm. The length L₄ of the second downstream side channel was set to 32.6 mm, and the length L of the cross channel was set to 214 μm. A width of each channel was set to 104 μm, and a depth of each channel was set to 48 μm.

A separation medium was injected into each port of the microchip, and channels were filled with the separation medium. Subsequently, the sample solution immediately after slow cooling was injected into the port #1, and then voltage at each port was applied as shown in Table 1 (introduction step). By the above, the sample solution was introduced into the sample introduction channel, and the sample solution in the cross channel was prevented from diffusing to the second direction upstream side and downstream side. Potential and potential gradient of each port and each channel at this time are also shown in Table 1.

TABLE 1

| | | Applied voltage | Potential | Potential gradient | Direction of | Movement direction |
|---|---|---|---|---|---|---|
| Port | Channel | V (V) | P (V) | E (V/cm) | current | of sample |
| #1 | First upstream | 280 | 280 | 210 | From cross channel | From port |
| | side channel | | | | to port | to cross channel |
| #2 | First downstream | 530 | 530 | 470 | From port | From cross channel |
| | side channel | | | | to cross channel | to port |
| #3 | Second upstream | 320 | 320 | 160 | From cross channel | From port |
| | side channel | | | | to port | to cross channel |
| #4 | Second downstream | 0 | 0 | 110 | From cross channel | From port |
| | side channel | | | | to port | to cross channel |
| — | Cross channel | — | 357 | — | — | — |

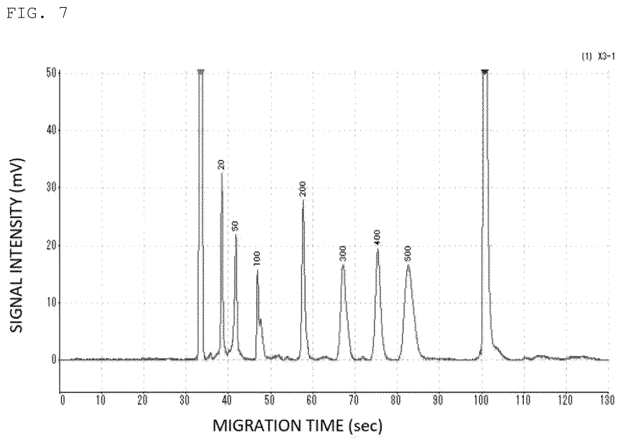

Next, applied voltage of each port was changed as shown in Table 2, and separation analysis of the sample was performed (separation step). The electropherogram at this time is illustrated in FIG. 7.

TABLE 2

| | | Applied voltage | Potential | Potential gradient | Direction of | Movement direction |
|---|---|---|---|---|---|---|
| Port | Channel | V′ (V) | P′ (V) | E′ (V/cm) | current | of sample |
| #1 | First upstream | 270 | 270 | 170 | From port | From cross channel |
| | side channel | | | | to cross channel | to port |
| #2 | First downstream | 270 | 270 | 170 | From port | From cross channel |
| | side channel | | | | to cross channel | to port |
| #3 | Second upstream | 0 | 0 | 580 | From cross channel | From port |
| | side channel | | | | to port | to cross channel |
| #4 | Second downstream | 1020 | 1020 | 250 | From port | From cross channel |
| | side channel | | | | to cross channel | to port |
| — | Cross channel | — | 210 | — | — | — |

As is clear from FIG. 7, a peak of RNA of 20 to 500 bases is clearly observed, and it can be seen that the electrophoresis method of Example 1 can perform analysis with excellent separability.

### Example 2

The electrophoresis method of the present invention was performed in the same manner as in Example 1 except that 680 bp DNA and a 25 bp ladder were used as a sample, and a difference (E₃−E₄) between the potential gradient E₃ on the second direction upstream side and the potential gradient E₄ on the second direction downstream side was changed in a range of 20 to 70 V/cm (specifically, 70, 50, and 20 V/cm) during the introduction step, to obtain an electropherogram.

### Comparative Example 1

An electropherogram was obtained by performing the electrophoresis method of a comparative example in the same manner as in Example 2 except that a difference (E₃−E₄) between the potential gradient E₃ and the potential gradient E₄ was changed in a range of −40 to 0/cm (Specifically, 0, −10, −30, and −40 V/cm).

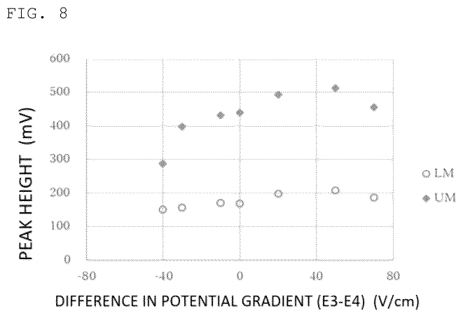

In the electropherograms obtained in Example 2 and Comparative Example 1, a height of a peak of DNA of a shortest chain (base length 25 bp) and a height of a peak of DNA of a longest chain (base length 680 bp) were measured, and results of these are shown in FIG. 8. In FIG. 8, a peak height of DNA of a shortest chain is indicated by a plot of “LM”, and a peak height of DNA of a longest chain is indicated by a plot of “UM”. From FIG. 8, it can be seen that as potential gradient on the second direction upstream side is higher, that is, in Example 2 in which E₃>E₄, a height of a peak is high and sensitivity is excellent in both LM and UM as compared with Comparative Example 1 in which E₃≤E₄.

### Example 3

An electropherogram was obtained by performing the electrophoresis method of the present invention in the same manner as in Example 1 except that 680 bp DNA and a 25 bp ladder were used as a sample, and both the potential gradient E′₁ on the first direction upstream side and the potential gradient E′₂ on the first direction downstream side were changed in a range of 140 to 200 v/cm during the separation step.

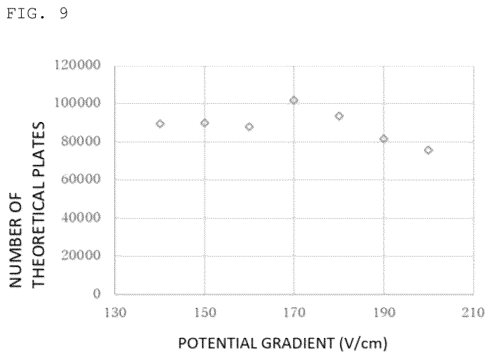

In the electropherogram obtained in Example 3, the number of theoretical plates was calculated by focusing on a peak of DNA of a longest chain (base length 680 bp). Results of these are shown in FIG. 9. From FIG. 9, it can be seen that in a case where potential gradient is 165 to 185 v/cm, the number of theoretical plates is particularly excellent, and separability is further excellent.

What is claimed is:

1. A microchip electrophoresis method comprising:
in order,
a preparation step of preparing a chip for electrophoresis including a sample introduction channel extending in a first direction and a sample separation channel extending in a second direction intersecting the first direction;
a filling step of filling the sample introduction channel and the sample separation channel with a separation medium for electrophoresis;
an introduction step of introducing a sample into the sample introduction channel and applying voltage to the sample introduction channel so as to generate potential gradient in the first direction in the sample introduction channel, and thus filling the entire sample introduction channel with the sample; and
a separation step of separating a target substance in the sample by applying voltage into the sample separation channel so that potential gradient is generated in the second direction in the sample separation channel,
wherein the sample introduction channel includes:
an upstream side channel extending in the first direction;
a cross channel extending in the second direction from a first direction downstream side end portion of the upstream side channel; and
a downstream side channel extending in the first direction from a second direction downstream side end portion of the cross channel,
the sample separation channel shares the cross channel in a middle thereof, and
in the introduction step, voltage is applied to the sample separation channel such that potential in the cross channel is higher than potential on the second direction upstream side and the second direction downstream side of the cross channel, and such that potential gradient on the second direction upstream side of the cross channel is larger than potential gradient on the second direction downstream side of the cross channel.

2. The microchip electrophoresis method according to claim 1, wherein in the introduction step, a difference between potential gradient on a second direction upstream side of the cross channel and potential gradient on a second direction downstream side of the cross channel is set to 20 V/cm or more and 70 V/cm or less.

3. The microchip electrophoresis method according to claim 1, wherein
the target substance is short-chain RNA, and
the microchip electrophoresis method further comprises a thermal denaturation step to perform heating so that the target substance becomes straight-chain before the introduction step.

4. The microchip electrophoresis method according to claim 1, wherein the separation medium for electrophoresis contains a water-soluble cellulose derivative and sugar alcohol.
